(19) United States
(12) Patent Application Publication
(10) Pub. No.: US 2018/0028261 A1
(43) Pub. Date: Feb. 1, 2018

(54) DEVICE AND METHOD FOR ASSISTING IN TISSUE ABLATION

(71) Applicant: KONINKLIJKE PHILIPS N.V., EINDHOVEN (NL)

(72) Inventors: YINAN CHEN, SHANGHAI (CN); JUNBO LI, SHANGHAI (CN); THOMAS SHU YIN TANG, MARKHAM (CA); GLADYS TSZ LING CHAN, TORONTO (CA); NICK QIU, Toronto (CA)

(21) Appl. No.: 15/550,900

(22) PCT Filed: Feb. 1, 2016
(86) PCT No.: PCT/EP2016/052011

§ 371 Date: Aug. 14, 2017

(30) Foreign Application Priority Data
Feb. 17, 2015 (CN) PCT/CN2015/000109
Apr. 7, 2015 (EP) 15162544.9

(51) Int. Cl. A61B 18/14; A61B 8/08; A61B 34/10; A61B 34/00

(52) U.S. Cl. CPC A61B 18/1477; A61B 34/25; A61B 8/085; A61B 34/10; A61B 8/481; A61B 2018/00577

(57) ABSTRACT

The embodiments disclose a device, method and system for assisting in the determination of one or more ablation regions for covering a target region to be ablated. The device comprises a processor configured to receive vascular structure information of vessels inside the target region and derive a parametric map for the target region based on the received vascular structure information, each value in the parametric map indicating a metric of a corresponding voxel of the target region to be inside the one or more ablation regions. The parametric map serves to assist in the determination of the one or more ablation regions.

### TECHNICAL FIELD

Example embodiments of the present disclosure generally relate to medical imaging, and more particularly to assisting in tissue ablation using medical imaging data.

### BACKGROUND

Ablation is one option for cancer treatment. In spite of recent advances in cancer therapy, treatment of primary and metastatic tumors of the abdomen remains a significant challenge. Hepatocellular carcinoma (HCC) for example is one of the most common malignancies encountered throughout the world (e.g., >1 million cases per year). In the United States alone, 1 in 153 individuals will develop HCC with reported 5-year survival rates of less than 15%.

For both primary liver cancer and hepatic metastases, liver resection (partial hepatectomy) is the current preferred option in patients having confined disease. In selected cases of early HCC, total hepatectomy with liver transplantation may also be considered. Unfortunately, less than 25% of patients with primary or secondary liver cancer are candidates for resection or transplantation, primarily due to tumor type, location, or underlying liver disease. Consequently, increasing interest has been focused on ablative approaches for the treatment of unresectable liver tumors. Rather than extirpation, this technique uses complete local in situ tumor destruction. A variety of methods have been employed to locally ablate tissue. Radio frequency ablation (RFA) is the most commonly used technique, but other techniques are also used, including ethanol injection, cryo-therapy, irreversible electroporation, and microwave ablation.

The RFA procedure is performed by placing an ablation device such as a needle within the target region, being an area to be ablated, such as a tumor in the liver parenchyma. Electrodes at the tip of the needle create heat, which is conducted into the surrounding tissue, causing coagulative necrosis at temperatures between 50° C. and 100° C. within a certain range. In addition to increasing the number of patients eligible for curative therapy of liver cancer in unresectable patients, local tissue ablation has a significant advantage as ablation may be performed using a minimally invasive approach, including percutaneously and laparoscopically. Since the ablation region of a single needle is limited, additional needles will be used or alternatively the needle will be repositioned so as to generate more than one ablation regions to cover a relatively large percentage of the target region. The success of the procedure partly depends on the placement of the needle. Different placements may have different results.

The clinicians often rely on intra-operative imaging techniques, such as ultrasound, to manually determine one or more locations inside the target region to place the needle, resulting in one or more ablation regions. Thus, the determined one or more needle locations and the resulted one or more ablation regions are highly dependent on the skills and experiences of the individual clinicians.

Recently, computer-assisted ablation planning has been proposed to assist the ablation procedure, particularly with respect to planning one or more locations to place the needle so as to cover the whole target region. Some existing computer-assisted ablation planning performs the planning based on the shape and size of the target region with the purpose of maximizing the overlap of the ablation regions produced by performing the ablation at the one or more locations and the target region. In some other existing approaches, additional factors are taken into account to assist in the ablation procedure. In US2009/221999A1, US2014/296842A1, US2011/201925 and US2014/136174A1, vessels adjacent to or in the vicinity of the tumor to be ablated, which serve as local heat sink in thermal ablation procedure, are segmented and properly taken into account to simulate the heat transport phenomena, temperature map or heat diffusion. In WO2008/132664A2, it is proposed to compute the risk relating to injuring an anatomical structure by a medical device such as an ablation device.

### SUMMARY

Therefore, it is an object to provide a device, a method and/or a system for assisting a determination of one or more ablation regions which are intended to cover a target region to be ablated.

According to one aspect of the embodiments, there is provided a device for assisting in the determination of one or more ablation regions for covering a target region to be ablated. The device comprises a processor configured to: receive vascular structure information of vessels inside the target region; and derive a parametric map for the target region based on the received vascular structure information, each value in the parametric map indicating a metric of a corresponding voxel of the target region to be inside the one or more ablation regions. The parametric map serves to assist in the determination of the one or more ablation regions, for example, either by being presented to the clinicians and/or by being provided for further processing. In some embodiments, the target region to be ablated can be the same as tissue volume, such as a tumor volume, to be ablated. In some other embodiments, the target region to be ablated can be a geometric combination of tissue volume to be ablated and a predetermined safety margin, typically 5 mm to 10 mm, surrounding the boundary of the tissue volume to be ablated, which is known as planned target volume (PTV).

In some embodiments, a higher value indicates that it is more desirable for having the corresponding voxel to be inside the one or more ablation regions as compared to another voxel with a lower value. Alternatively, a lower value may indicate that it is more desirable for having the corresponding voxel to be inside the one or more ablation regions as compared to another voxel with a higher value. In other words, each value in the parametric map indicates the cost of the corresponding voxel of the target region for being inside the one or more ablation regions or the desirability that the corresponding voxel of the target region is inside the one or more ablation regions.

In comparison with conventional images which represent the vascularity information at each voxel, the parametric map directly provides information on whether it is desirable to have the voxel inside the ablation regions.

Furthermore, as completely different from the aforementioned existing approaches wherein vessels surrounding the target region to be ablated, which are typically large vessels, are segmented so as to study the heat dissipation, the present invention disclose receiving vascular structure information of vessels inside the target region to be ablated, which are typically micro vessels, and derives the parametric map on the basis of vascular structure information of the vessels inside the target region. In case of tumor ablation procedure, vessels inside the tumor to be ablated is known as intratumoral vessels.

In one embodiment, the device further comprises a first user interface configured to present the derived parametric map. For example, the device further comprises an image encoder configured to produce corresponding display values for the values of the parametric map; and the first user interface is configured to display the display values as a parametric image. The image encoder can be further configured to encode the values of the parametric map with distinctive coloring or shading.

In one embodiment, the processor is further configured to determine position of the one or more ablation regions based on the derived parametric map.

In one embodiment, the processor is further configured to identify one or more risky regions in the target region, based on the parametric map and a value threshold for a voxel to be in the risky region. The one or more risky regions serve to assist in the determination of the one or more ablation regions by being presented to the clinicians and/or by being provided for further processing. A risky region is known as a region which, if it is not ablated, may result in risk for the subject and thus is undesirable to be part of ablative residual regions.

In one embodiment, the value threshold is derived based on the parametric map and a predetermined ablation coverage rate.

In one embodiment, the determination of the one or more ablation regions is further based on one or more of: part of the target region which is not covered by the one or more ablation regions; part of the one or more ablation regions which is not covered by the target region; and part of the one or more ablation regions which is overlapped with a predetermined critical region.

In one embodiment, the device further comprises a second user interface, wherein the second user interface is configured to receive at least one of the following user inputs: a user input for indicating a number or a maximum number of entry points for the one or more ablation regions; a user input for indicating position of one or more entry points for the one or more ablation regions; a user input for indicating a number or a maximum number of the one or more ablation regions; a user input for indicating position of the one or more ablation regions; and the processor is further configured to determine the position of the one or more ablation regions, considering the derived parametric map and the received at least one user input.

In one embodiment, the processor is further configured to: assess the one or more ablation regions, considering the derived parametric map; derive an indicator from the assessing result; and output the derived indicator via a third user interface.

In one embodiment, the vascular structure information of the target region comprises an angiography image of the target region.

According to another aspect of the embodiments, there is provided a method for assisting in a determination of one or more ablation regions for covering a target region to be ablated. The method comprises: receiving vascular structure information of vessels inside the target region; and deriving a parametric map for the target region, based on the received vascular structure information, each value in the parametric map indicating a metric of a corresponding voxel of the target region to be inside the one or more ablation regions, wherein the parametric map serves for assisting in the determination of the one or more ablation regions.

According to a third aspect of the embodiments, there is provided a system for assisting in a determination of one or more ablation regions for covering a target region to be ablated. The system comprises: an imaging component configured to generate vascular structure information of vessels inside the target region; a processor in communication with the imaging component, the processor being configured to: receive the vascular structure information of vessels inside the target region; and derive a parametric map for the target region, based on the received vascular structure information, each value in the parametric map indicating a metric of a corresponding voxel of the target region to be inside the one or more ablation regions, wherein the parametric map serves to assist in the determination of the one or more ablation regions.

### BRIEF DESCRIPTION OF THE DRAWINGS

The technology will now be described, by way of example, based on embodiments with reference to the accompanying drawings, wherein:

FIG. 1 illustrates a system for assisting in the determination of one or more ablation regions for covering a target region to be ablated, in accordance with one or more aspects set forth herein;

FIG. 2 illustrates a block diagram of a component of the system for assisting in the determination of one or more ablation regions for covering a target region to be ablated, in accordance with one or more aspects set forth herein;

FIG. 3 illustrates a flowchart of a method of assisting in the determination of one or more ablation regions for covering a target region to be ablated, in accordance with one or more aspects set forth herein;

FIG. 4 illustrates a flowchart of another method of assisting in the determination of one or more ablation regions for covering a target region to be ablated, in accordance with one or more aspects set forth herein;

FIG. 5 illustrates determined, expected ellipsoid-shaped ablation regions in accordance with one or more aspects set forth herein;

FIG. 6 illustrates some graphics based on one metric or assisting in the determination of one or more ablation regions for covering a target region to be ablated, in accordance with one or more aspects set forth herein;

FIG. 7 illustrates a flowchart of imaging during ultrasound data acquisition, in accordance with one or more aspects set forth herein;

FIG. 8 illustrates some graphics output by the system for assisting in the determination of one or more ablation regions for covering a target region to be ablated, in accordance with one or more aspects set forth herein;

FIG. 9 illustrates determined, ellipsoid-shaped ablation regions in accordance with one or more aspects set forth herein.

### DETAILED DESCRIPTION

Embodiments herein will be described in detail hereinafter with reference to the accompanying drawings, in which embodiments are shown. The embodiments described herein may, however, be embodied in many different forms and should not be construed as being limited to the embodiments set forth herein. The elements of the drawings are not necessarily drawn to scale relative to each other. Like numbers refer to like elements throughout.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood. It will be further understood that terms used herein should be interpreted as having a meaning that is consistent with their meaning in the context of this specification and the relevant art and are not be interpreted in an idealized or overly formal sense unless expressly so defined herein.

The present technology is described below with reference to block diagrams and/or flowchart illustrations of methods, apparatus (systems) and/or computer program products according to the present embodiments. It is understood that blocks of the block diagrams and/or flowchart illustrations, and combinations of blocks in the block diagrams and/or flowchart illustrations, may be implemented by computer program instructions. These computer program instructions may be provided to a processor, controller or controlling unit of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

Embodiments herein will be described below with reference to the drawings.

Hepatocellular carcinoma (HCC), the most common primary liver tumor, is notoriously resistant to systemic therapy, and often recurs even after aggressive local therapies. HCC is dependent upon angiogenesis—the growth of new capillary blood vessels—to supply tumors with oxygen and nutrients. Tumor angiogenesis is stimulated by proteins called growth factors. The primary angiogenesis-stimulating growth factor is called vascular endothelial growth factor (VEGF). Most malignant tumors produce large amounts of VEGF and other growth factors to create a dedicated blood supply for the tumor. A hallmark of new vessel formation in tumors is their structural and functional abnormality. This leads to an abnormal tumor microenvironment characterized by low oxygen tension. The liver is perfused by both arterial and venous blood and the resulting abnormal microenvironment selects for more-aggressive malignancies. The vascularity and morphologic changes of intratumoral vessels reflect the different stages of the tumor progression.

To understand the hemodynamics and angiogenesis of HCC is important for the precise imaging diagnosis, treatment and follow-ups, because there is an intense correlation between hypervascular properties and pathophysiology. Tumors often encounter hypoxic conditions during their growth. Under such conditions, hypoxia inducible factor-1α (HIF-1α) promotes the transcriptional activity of angiogenesis related molecules such as VEGF and erythropoietin by affecting the hypoxia response element and HIF-1α. It was reported that hepatocellular areas around the portal tracts in dysplastic nodules, including those with hepatic sinusoidal capillarization and unpaired arterials, were strongly positive for HIF-1α, whereas this molecule was faintly expressed in the surrounding livers. Cytoplasmic overexpression and intra-nuclear expression of HIF-1α, a more increased expression pattern, were also observed in HCC, suggesting that cytoplasmic HIF-1α might have been moved into the nuclei in activated HCC cells. HIF-1α is involved in the upregulation of genes harboring the hypoxia response element such as VEGF, suggesting that increased expression of HIF-1α in the areas around the portal tracts of dysplastic nodules may be responsible for increased expression of VEGF and its receptor followed by sinusoidal capillarization and increased numbers of unpaired arteries in dysplastic nodules and also in the angiogenesis in HCC. These expressions gradually spread into the entire nodule in accordance with the elevation of the grade of malignancy of the nodules. Hepatic arteriography using CT, MR or Ultrasound imaging system, provides a well-differentiated focus demonstrating a faint enhancement and this portion reveals more expression of sinusoidal capillarization and unpaired arteries than that in the surrounding high-grade dysplastic nodule. It indicates the gradual increase of angiogenesis during multi-step hepatocarcinogenesis, which eventually evolves to advanced HCC through the repeated substitution of malignant and poorly differentiated tissue in lesion. Sequential changes in intranodular hemodynamics during evolution to HCC include degeneration of preexisting hepatic arteries and portal veins and a gradual increase in neo-vascularized arteries.

The development of microbubble ultrasound contrast agents has overcome some of the limitations of conventional B-mode and Doppler ultrasound techniques for the liver and enables the display of the parenchymal microvasculature. Contrast-enhanced ultrasonography (CEUS) modes cancel the linear ultrasound signal from tissue and utilize the nonlinear responses from the microbubbles. The enhancement patterns of lesions can be studied during all vascular phases (arterial, portal venous, late and post vascular phases), in a similar fashion to contrast enhanced CT and contrast enhanced magnetic resonance imaging but in real time and under full control of the sonographer.

Contrast-enhanced ultrasound (CEUS) has recently been introduced and is recommended in daily routine under many circumstances, mainly in the detection and characterization of focal liver lesions. Recently, guidelines for the use of CEUS have been published to improve the management of patients. The European Federation of Societies for Ultrasound in Medicine and Biology (EFSUMB) guidelines are based on comprehensive literature surveys including results from prospective clinical trials. The use of contrast agents in the liver is possible for different purposes: detection of liver tumors, characterization of liver tumors (benign versus malignant), monitoring local ablative treatment, and imaging hepatic vessels and measuring the hepatic transit time.

With the development of ultrasonographic contrast agents and contrast-specific imaging techniques, CEUS has greatly improved the ability to visualize blood flow perfusion of focal liver lesions. Micro-flow imaging (MFI) realizes the angiography imaging by using ultrasound modality. It is a new contrast-enhanced ultrasonographic modality using low-mechanical index (MI) CEUS and an accumulative imaging technique to show blood vessels after a flash with high-transmission power ultrasound exposure. First, high-transmission power ultrasound destroys the microbubbles in the scan volume, and then replenishment of the microbubbles into the scan volume is observed by using a harmonic imaging mode at low transmission power. The number of microbubbles in the small vessels is still small at the moment immediately after high-transmission power ultrasound exposure. The small vessels are therefore not visualized on conventional contrast imaging. Maximum-holding images are more sensitive in detecting microbubbles in the circulation than conventional contrast images and very efficient at visualizing micro-vessels, even though the number of microbubbles in these vessels is very small or flow is very slow. Therefore, the principle of MFI is that just after a flash with high transmission power, the ultrasonography system starts maximum-holding image processing to trace the position of the moving bubbles with high sensitivity and to display the micro-vessels. The constructed micro-vessels in the liver are finally covered by microbubbles inflowing into the sinusoid.

HCC is generally a hypervascular tumor. The majority of liver lesions display homogeneous or heterogeneous hyper-enhancement in the arterial phase, but intratumoral vessels are not always shown in the arterial phase from the untargeted CEUS. MFI distinctly delineated the vascular architecture in detail with high confidence. Microvascular changes depicted by MFI correlated well with the pathologic differentiation of HCC.

Thermal ablation is a minimally invasive image-guided therapy for the safe and effective treatment of localized nodule disease, mainly including radiofrequency ablation (RFA) and microwave ablation (MWA). A computer-assisted thermal ablation planning tool is a time-efficient product to geometrically analyze a reasonable overlap area of the composite ablations with the tumor volume in 3D. From a computer modelling perspective, a complete necrosis rate of the arbitrary-shaped planned target volume (PTV) is important but not the only metric. For example, PTV can be a geometric combination of tumor volume and a 5 mm-10 mm user-configured safety margin surrounding the tumor boundary. Successful thermal planning allows a user to explore coverage of a large arbitrary-shaped PTV with a minimum number of ablations while simultaneously minimizing collateral damage. Not in a limited manner, but more generically, the estimate of the ablation numbers is based on several considerations, e.g., size and shape of the tumor at each of the sites, estimated ablation size for a selected ablation probe, proximity of large blood vessels to the tumor sites, direction of the approach based on the skin entry points. Thermal ablation treatments for large tumors have shown a higher local recurrence rate, possibly due to lack of complete coverage of the arbitrary-shaped planned target volume. The ideal situation is a 100% coverage percentage of the PTV with sufficient ablations, i.e. no residual at all; however, it is unfortunately inevitable in some cases if damage avoidance of critical anatomic structures, such as gall bladder, intestine, vessel drainage system, are taken into account. In other words, it is unrealistic to achieve a 100% ablation coverage rate in complex treatment conditions due to the multi-parameter trade-off.

Besides, it is not possible in practice to achieve a 100% ablation coverage rate also due to: a) too many ablations and causal needle trajectories for a big lesion will result in a high rate of complications for a patient with a coagulopathy; b) a good planning algorithm should proceed a trade-off between the ablation coverage percentage and collateral critical structure damage; c) although the computer-assisted planning is perfect in some cases, the actual needle trajectory always has a bias with respect to the planned one during a human operation. Therefore, residual, i.e. anything in the output of the automatic planning process or the realistic intraoperative process, is inevitable. Generally, the coverage percentage of the target is set to around 90%-95%, resulting in a 5%-10% residual tolerance.

The current computer-assisted planning algorithm is only based on the morphological structure of the composite ablations for the purpose of maximizing the overlap of the PTV. The restraint for the algorithm optimization during multi-iteration is the minimal acceptance of the ablative residual or collateral damage in voxels. It is clear that the ablation residual will entail the possibility of local recurrence. Recurrence highly correlates to poorly differentiated tissue. If the residual area exactly contains the poorly differentiated tissue with a high density of the intratumoral vascularity, the situation is extremely risky.

In this invention, we try to improve the thermal ablation planning by searching a more optimal area of the composite ablations where the ablative residual has low probability of containing the micro-vessel structure. The benefit will be a reoccurrence reduction in spite of the ablative residual.

Ablation devices, such as an elongated slender probe, are typically inserted into a tumor, lesion, or other tissue to be ablated, and the probe tip is heated using a high radio frequency in order to heat the surrounding tissue to a temperature sufficient to kill cells therein, often considered to be 50 degrees Celsius. Although the present application primarily describes radio frequency (RF) ablation techniques, which can be used in many locations, including liver, kidney, breast, lung and others, it will be understood that cryoablation, microwave, and other ablation and treatment procedures can be planned similarly.

An ablation zone is typically located relative to the probe tip and is spheroid or ellipsoid in shape, noting that a sphere is an ellipsoid with equal a, b, c axes. When a tumor is larger than the ablation region for a given probe size, a surgeon selects more than one probe position to generate a plurality of ablated regions that overlap to cover the entire tumor mass. A typical ablation process involves defining the target region, inserting the probe into the desired location, and applying power to the probe for about 15 minutes, causing the probe tip to heat up.

A planned target volume (PTV) is defined that envelopes the entire tumor mass as well as a buffer region (e.g., typically one centimeter or so) around the tumor. This ensures ablation of all tumor cells and microscopic tumor cells, found in the buffer zone, in order to mitigate recurrence of the tumor. As indicated above, some example embodiments may enable the provision of a mechanism by means of which ablation regions are planned automatically or semi-automatically on the basis of machine-executed analysis of ultrasound data of a liver. In some cases, the data may be obtained by real-time imaging modalities such as preferably ultrasound.

It is noted that the embodiments described herein are not limited to just liver, or kidney, breast, lung; it will be appreciated by one skilled in the art that the embodiments described herein are applicable to all kinds of ablation plans.

The volume may be “grown” by a desired distance so that the tumor plus margin are included in the resulting volume. Whenever the word “tumor” is used herein, particularly regarding optimization, it is assumed to mean the “Planned Target Volume” (PTV), which covers the specified tumor plus safety margin that together are intended for full coverage.

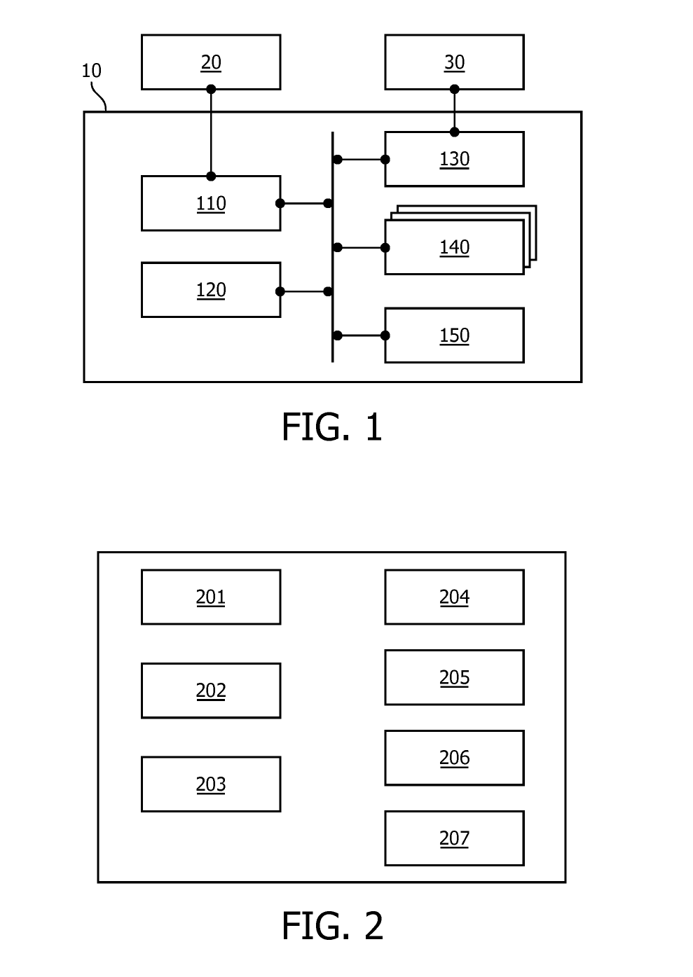

FIG. 1 illustrates a system for assisting in the determination of one or more ablation regions for covering a target region to be ablated, in accordance with one or more aspects set forth herein. In this example, the ultrasound system is embodied as a computer controlled device. Thus, for example, the ultrasound system may include an imaging component 20 and an ablation component 30.

The ablation component 30 in one embodiment is an RF ablation system, which includes a power source, a radio frequency generator, a probe operatively coupled thereto, etc., as well as any other suitable component to facilitate inserting the probe into a tumor mass and heating the tumor mass to a temperature sufficient to kill tumor cells (e.g., approximately 50 degrees Celsius) within a region relative to the probe tip.

The imaging component 20 may be an imaging device configured to obtain data of a liver of a subject. The data collectable may be captured noninvasively by obtaining data using an ultrasound probe that remains external to the body, but measures ultrasound waves that pass through and/or reflect off of various body parts. In an example embodiment, the imaging component 20 may be embodied as or include real-time imaging modalities such as, preferably, ultrasound. Ultrasound in particular may provide a relatively low cost, low power, portable modality. However, the imaging component 20 is not limited to just ultrasound.

The imaging component 20 may provide data to the ablation planner 10, which may be configured to receive and process data captured by the imaging component 20 in order to generate parametric maps that may be used for planning ablation regions. In some cases, the ablation planner 10 may receive the data in real time (or near real time) directly from the imaging component 20. However, in other cases, data from the imaging component 20 may be stored first, and may thereafter be retrieved from storage before being analyzed by the ablation planner 10.

As shown in FIG. 1, the ablation planner 10 may include, or otherwise be in communication with, processor 110 that is configurable to perform actions in accordance with example embodiments described herein. Therefore, for example, at least some of the functions attributable to the ablation planner 10 may be carried out, or otherwise instructed, by the processor 110. The processor 110 may therefore provide the hardware for hosting software to configure the system for machine learning and machine driven analysis techniques consistent with example embodiments. Ablation region planning or assisting in ablation region planning may then be accomplished using the processor 110.

The processor 110 may be configured to perform data processing, control function execution and/or other processing and management services according to an example embodiment of the present invention. In some embodiments, the processor 110 may be embodied as a chip or chip set. In other words, the processor 110 may comprise one or more physical packages (e.g., chips) including materials, components and/or wires on a structural assembler (e.g., a baseboard).

In an example embodiment, the processor 110 may include one or more instances of a processor 110 and memory 150 that may be in communication with, or otherwise control, a second interface 130 and, in some cases, a user interface (UI) 140. As such, the processor 110 may be embodied as a circuit chip (e.g., an integrated circuit chip) configured (e.g., with hardware, software or a combination of hardware and software) to perform operations described herein.

The user interface 140 may be in communication with the processor 110 to receive an indication of a user input at the user interface 140 and/or to provide an audible, visual, mechanical or other output to the user. As such, the user interface 140 may include, for example, a display, one or more buttons or keys (e.g., function buttons), and/or other input/output mechanisms (e.g., keyboard, microphone, speakers, cursor, joystick, lights and/or the like). The user interface 140 may be embodied as more than one independent hardware components. The user interface 140 may display information indicating an identity or certain characteristics of a data set (e.g., including raw RF data or results of analyzing the raw RF data) being processed by the ablation planner 10. The characteristics of the data set may then be processed and information associated therewith may be presented on a display of the user interface 140, based on instructions executed by the processor 110 for the analysis of the data according to prescribed methodologies and/or algorithms. Moreover, in some cases, the user interface 140 may include options for selection of one or more reports to be generated based on the analysis of a given data set.

The first interface 110 may include one or more interface mechanisms for enabling communication with an external device, i.e., the imaging component 20, or internal functional components of the ablation planner 10. In some cases, the first interface 110 may be any means such as a device or circuitry embodied in either hardware, or a combination of hardware and software, that is configured to receive and/or transmit data from/to devices in communication with the processor 110.

The second interface 130 may also include one or more interface mechanisms for enabling communication with another external device, i.e., the imaging component 20, or internal functional components of the ablation planner 10. In some cases, the second interface 130 may be any means such as a device or circuitry embodied in either hardware, or a combination of hardware and software, that is configured to receive and/or transmit data from/to devices in communication with the processor 110.

In an example embodiment, the memory 150 may include one or more non-transitory memory devices such as, for example, one or more volatile and/or non-volatile memories that may be either fixed or removable. The memory 150 may be configured to store information, data, applications, instructions or the like for enabling the ablation planner 10 to carry out various functions in accordance with example embodiments of the present invention. For example, the memory 150 could be configured to buffer input data for processing by the processor 110. Additionally or alternatively, the memory 150 could be configured to store instructions for execution by the processor 110. As yet another alternative, the memory 150 may include one or more databases that may store a variety of data sets such as data obtained from the imaging component 20, or conventional navigation information data from electromagnetic tracking systems, and/or the like, to be employed for the execution of example embodiments. Among the contents of the memory 150, applications may be stored for execution by the processor 110 in order to carry out the functionality associated with each respective application. In some cases, the applications may include instructions for control of the ablation planner 10 to generate a parametric map for a target region, each value in the parametric map indicating a metric of a corresponding voxel of the target region to be inside the one or more ablation regions and/or employ analytical tools for analyzing data to identify risky regions in view of vascular structure information of vessels inside the target region and analyze data therein to determine a target region to be ablated. In some cases, the applications may further include instructions for generating outputs and/or reports associated with analysis of patient data as described herein.

The processor 110 may be embodied in a number of different ways. For example, the processor 110 may be embodied as various processing means such as one or more of a microprocessor or other processing element, a coprocessor, a controller or various other computing or processing devices including integrated circuits such as, for example, an ASIC (application specific integrated circuit), an FPGA (field programmable gate array), or the like. In an example embodiment, the processor 110 may be configured to execute instructions stored in the memory 150 or otherwise accessible to the processor 110. As such, whether configured by hardware or by a combination of hardware and software, the processor 110 may represent an entity (e.g., physically embodied in circuitry) capable of performing operations according to example embodiments of the present invention while being configured accordingly. Thus, for example, when the processor 110 is embodied as an ASIC, FPGA or the like, the processor 110 may be specifically configured hardware for conducting the operations described herein. Alternatively, as another example, when the processor 110 is embodied as an executor of software instructions, the instructions may specifically configure the processor 110 to perform the operations described herein.

In an example embodiment, the processor 110 may be embodied as, include or otherwise control the ablation planner 10. As such, in some embodiments, the processor 110 may be said to cause each of the operations described in connection with the ablation planner 10 by directing the ablation planner 10 to undertake the corresponding functionalities responsive to execution of instructions or algorithms configuring the processor 110 accordingly.

In an example embodiment, data captured in association with ultrasound scanning of the liver of a particular patient may be stored (e.g., in the memory 150) or passed directly to the ablation planner 10. Thereafter, the data may be processed by the ablation planner 10 to enable the processor 110 to process the data in real time (or near real time) or to process the data as the data is extracted from memory.

In one embodiment, the imaging component 20 is configured to obtain data for analysis or processing, such as an angiography image with vascular structure information of vessels inside a target region, sometimes maybe by way of ultrasound, CT or MRI (for example, MFI image, a kind of contrast based ultrasound image), which, or analyzing results of which, will serve to assist in the determination of one or more ablation regions for the target region to be ablated.

In an example embodiment, the processor 110 includes a data receiver 201, a parametric map deriver 202, and an output controller 207. Additionally, the processor 110 may further include one or more of the following: a position determiner 203, a risky region identifier 204, an assessor 205, and an indicator deriver 206, shown in FIG. 2.

The data receiver 201 is configured to receive data for analysis or processing, such as an angiography image with vascular structure information of vessels inside a target region, sometimes maybe by way of ultrasound, CT or MRI (for example, MFI image, a kind of a contrast based ultrasound image). The data receiver 201 can also be configured to receive other data, such as user input.

The parametric map deriver 202 is configured to derive a parametric map for the target region, based on the obtained vascular structure information, each value in the parametric map indicating a metric of a corresponding voxel of the target region to be inside the one or more ablation regions in view of intratumoral vascularity restraint, wherein the parametric map is used to assist in the determination of the one or more ablation regions. In the case that the target region is a tumor, the value in the parametric map represents the intratumoral vascularity property, and thus it is also referred to as intratumoral vascularity property (IVP). The metric stands for an overall desirability, entailing vessel density, texture feature for indicating e.g. divergence of the vessel, morphology such as curving rate, etc., for a corresponding voxel of the target region to be inside or covered by the one or more ablation regions. As is known to a person skilled in the art, the higher the density of vessels inside the tumor, the lower the divergence of vessels inside the tumor is, or the more straight the vessels inside the tumor, the riskier the regions of the vessels are in relation to tumor growing. Therefore, voxels of these areas will be assigned a higher value, calculated from vessel density, texture feature, morphology, and/or other parameters indicating other features which are extracted from the angiography image, weighted by a factor respectively. In this way, the parametric map can assist as one of the restraints in an assessment metric for a safe residual area output.

It is noted that the parametric map can be shown to a user and help the user to easily recognize intratumoral vessels and then decide on the ablation regions manually. For example, User is allowed to manually set the desired ablation targets in a viewer where angiography image data and conventional B-mode image data are geographically fused together. This allows the user to visually judge the therapeutic validity and enables operability for the planned composite ablations on both the perspectives of tumor morphology and tumor peripheral structure analysis from a conventional B-mode image, and the intratumoral vascularity property from the angiography image.

Alternatively, the parametric map can also be made as input for the processor to automatically determine the position of one or more ablation regions. The position determiner 203 is configured to determine the position of one or more ablation regions considering the derived parametric map. For example, the position determiner 203 at first randomly generates three initial ablation regions, such as three ellipses, and then iteratively looks for three optimal ablation regions. That is, the number of ablation regions can be fixed. Alternatively, the position determiner 203 may start with only one ablation region; if it cannot meet the restrictions anyhow, the position determiner 203 may increase the number of ablation regions step by step and look for a number of optimal ablation regions until it achieves a satisfying result.

It is noted that variance in size of the ablation region can also be considered, as long as there are corresponding ablation needles. However, as the ablation probes are very expensive, one is deterred from using multiple probe sizes or configurations, in favor of attempting to ablate a tissue mass using a minimum number of probes; therefore, generally only one ablation probe is applied, i.e., size of one ablation region is normally fixed.

It is noted that a user may input some restrictions via the UI 140 to aid the position determiner 203 to determine positions of the one or more ablation regions. Such user input may be received by the data receiver 201 for processing. For example, the user may define the number of entry points, the position of the entry points, the number of total ablation regions, the number of ablation regions per entry point, etc. Thereafter, the position determiner 203 can determine the position of the one or more ablation regions considering the received at least one user input.

Optimization of the ablation regions involves assessment of the ablation regions in view of several restraints. Unlike the prior art, the residuals having a low risk of recurrence is an additional restraint, and due to the close relationship between recurrence and vascularity features, the parametric map may be used directly or indirectly for such a restraint.

The assessment of the ablation regions may be carried out by the assessor 205 and will be described later.

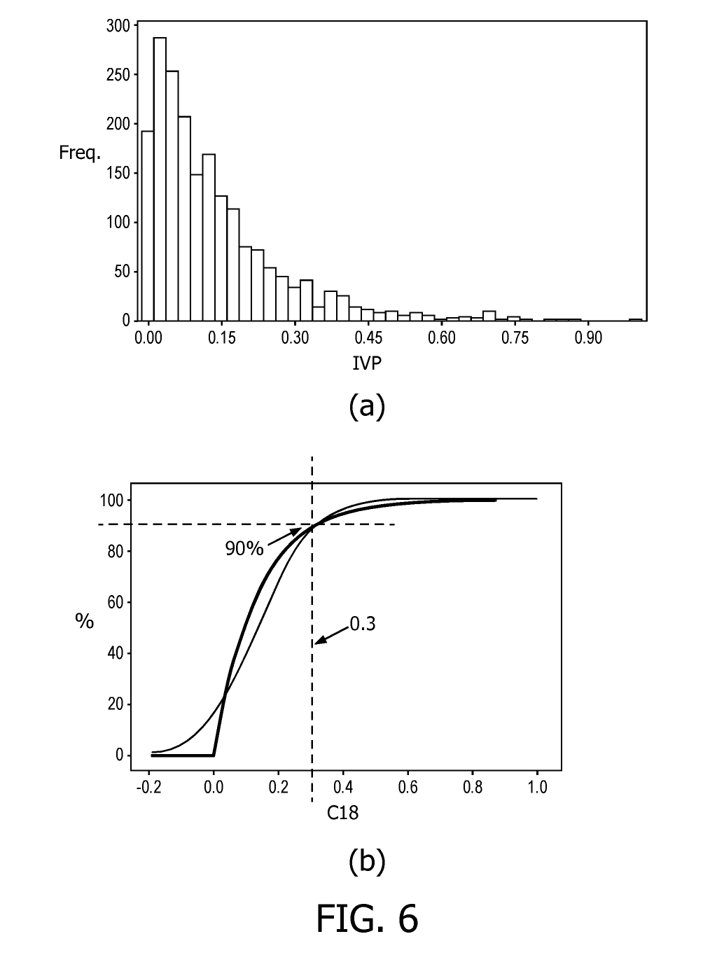

The risky region identifier 204 is configured to identify one or more risky regions for ablative residual in the target region, based on the parametric map and a value threshold for a voxel to be in the risky region, wherein the one or more risky regions serve for assisting in the determination of the one or more ablation regions, and wherein the value threshold is derived based on the parametric map and a predetermined ablation coverage rate. In one embodiment, in order to define the one or more risky regions for ablative residual, as described above, quantification of vessel severity in one or more target regions of angiography images (such as MFI images) is required for each voxel and represented by one or more image-based vascular feature variables, for example, vessel density, texture features, morphological features etc. The parametric map is thus derived. The watershed between risky and non-risky region is a threshold tᵣ which is adaptive to user-defined residual tolerance. In accordance with an embodiment, after the parametric map is derived, all the values (e.g. IVP values) of the parametric map are plotted in the format of a normalized histogram, as shown in FIG. 6 (a). The user specifies a minimum coverage rate, i.e. a minimum percentage of coverage pₚₜᵥ of the target region (e.g. planned target volume) for example 90%, and then the residual tolerance will be obviously obtained by 1−pₚₜᵥ, for example 1-90%=10%, and the threshold tᵣ can be derived from the normalized histogram where the frequency of the risk factor smaller than threshold tᵣ is only 1-pₚₜᵥ, for example 10%, and tᵣ is 0.3, as shown in FIG. 6 (b). Thereafter, the voxels with respective risk factors for ablasive residual smaller than threshold tᵣ are correspondingly defined as the non-risky regions. Conversely, the voxels with respective risk factors for ablasive residual not smaller than threshold tᵣ are correspondingly defined as the risky regions. The parametric map of the target regions is thus converted to a binary map by such threshold to separate the risky regions from the non-risky regions.

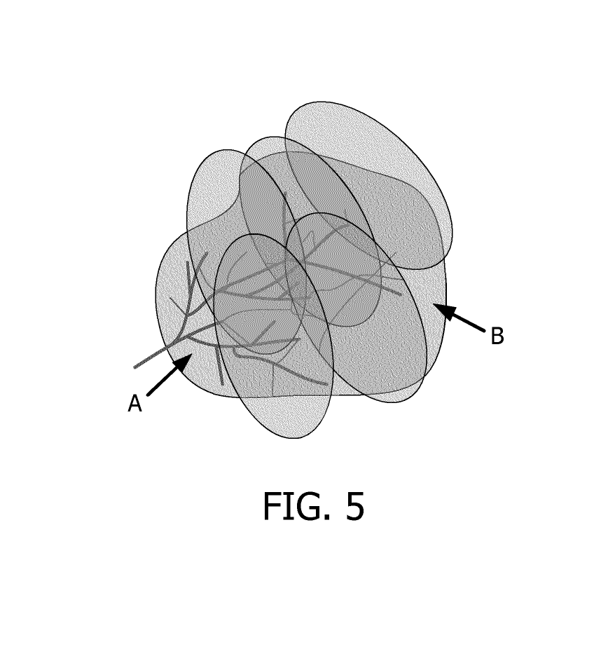

The assessor 205 is configured to assess the one or more ablation regions, considering the derived parametric map and other factors, such as ablation coverage rate or collateral damage, irrespective of whether they were determined manually by a user, or automatically by the position determiner 203. The assessment may apply a variety of algorithms, considering a variety of restraints. The assessment metric (also called cost function) for example can be a linear normalized combination of different restraints multiplied by the corresponding weightings. The restraints in the art normally comprise minimum accepted ablation coverage rate (namely the volume of the one or more ablation regions vs. the total volume of the target region), critical structure around the target region, collateral damage, and in the present invention, the overlap between ablation residual and the region being immersed with high intratumoral vascularity is formulized into voxels as one additional restraint in the assessment metric, which will be a penalty to avoid the ablation residual converging to a rich blood supply region. If the residual is inevitable in some cases with a large tumor or complex clinical situation, the improved planning algorithm is capable to deliver an ablation residual with low recurrence in a low-vascular or nonvascular area (A in FIG. 5) rather than a hypervascular area (B in FIG. 5).

In one embodiment, the user specifies a minimum desired ablation coverage rate, i.e., percentage of the target region (e.g. planned target volume) coverage pₚₜᵥ, the solutions for a sequentially increasing number of ablations are computed until the pₚₜᵥ value is achieved. The real-valued variables to be optimized are the coordinates of centers of the one or more ablations. For M ablations in 3D space, there are 3M real-valued variables (in three dimensions respectively) to be optimized. In accordance with some embodiments of the present invention, the determination of the one or more ablation regions is further based on one or more of:

part of the target region which is not covered by the one or more ablation regions (also referred to as residual region);

part of the one or more ablation regions which is not covered by the target region (also referred to as ablation regions outside the target region, which results in collateral damages); and

part of the one or more ablation regions which is overlapped with a predetermined critical region (namely part of the predetermined cirtial region which falls into the one or more ablation regions). The predetermined critical region comprises regions having critical structures which, desirably, are not to be ablated.

In some embodiments, the determination of the one or more ablation regions is further based on one or more of: the size of the residual region, the size of the part of the ablation regions outside the target region, the size of part of the predetermined critical region which falls into the ablation regions.

In an embodiment, by letting Nₚₜᵥ denote the number of voxels of the residual region, Nᵢᵥₚ denote the number of voxels of the residual region which are inside the risky regions, N_{cs} denote the number of voxels of the one or more ablation regions which are inside the critical region, N_{cd} denote the number of voxels of the one or more ablation regions which are not in the target region, the assessment metric or so-called cost function to minimize can be as follows:

$$costFuc = \frac{α {N}_{ptv} + β {N}_{ivp} + γ {N}_{cs} + {N}_{c d}}{α + β + γ}$$

wherein α, β, γ are the weightings associated with Nₚₜᵥ, Nᵢᵥₚ and N_{cs}, respectively. Using the knowledge of the clinical context allows us to attach a higher cost for lack of PTV coverage and for puncture into critical structures, a moderate cost for ablative residual in risky regions such as vessel aggressive areas, compared to collateral damage (i.e. ablated voxels outside the target region), namely α>γ>β≧100.

It is noted that the assessment metric value can be fed back to the position determiner 203 for it to optimize the final position of the one or more ablation regions.

The indicator deriver 206 is configured to derive an indicator from the assessing result. The indicator could simply be the assessment metric value, or an assessment metric level that the value belongs to. The indicator may also comprise ablation coverage rate and hint of collateral damage to other crucial tissue, etc.

The output controller 207 is configured to output the derived indicator via a third user interface.

The elements shown in FIGS. 1 and 2 are illustrated as separate elements. However, this is merely to indicate that the functionalities are separated. The elements can be provided as separate hardware devices. However, other arrangements are possible, such as the indicator deriver 206 and the output controller 207 can be physically combined into one unit. Any combination of the elements can be implemented in any combination of software, hardware, and/or firmware in any suitable location. For example, there could be one data receiver for receiving vascular structure information of vessels inside the target region and another data receiver for receiving user inputs configured separately.

Some of the elements may constitute machine-executable instructions embodied within a machine, e.g., readable medium, which when executed by a machine will cause the machine to perform the operations described. Besides, any of the elements may be implemented as hardware, such as an application specific integrated circuit (ASIC), Digital Signal Processor (DSP), Field Programmable Gate Array (FPGA) or the like.

Besides, it should be understood that the arrangements described herein are set forth only as examples. Other arrangements and elements (e.g., more UIs, more data receivers, etc.) can be used in addition to or instead of those shown, and some elements may be omitted altogether.

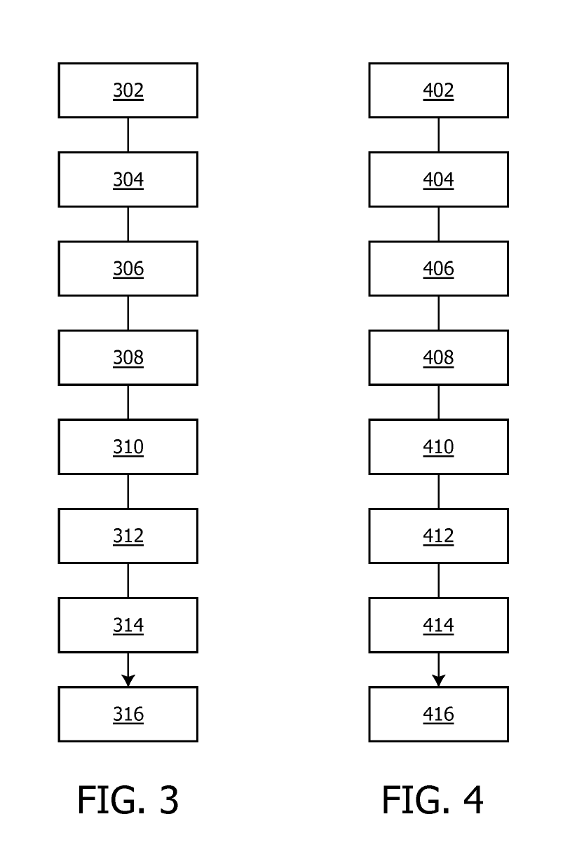

Functionalities and cooperation between those elements are described in detail with reference to FIGS. 3 and 4.

FIGS. 3 and 4 each illustrate a flowchart of a method for planning the one or more ablation regions of one example embodiment. It will be understood that each block of each flowchart, and combinations of blocks in each flowchart, may be implemented by various means, such as hardware, firmware, processor, circuitry and/or (an) other device(s) associated with execution of software including one or more computer program instructions. For example, one or more of the procedures described may be embodied by computer program instructions. In this regard, the computer program instructions which embody the procedures described above may be stored by a memory and executed by a processor. As will be appreciated, any such computer program instructions may be loaded onto a computer or other programmable apparatus (e.g., hardware) to produce a machine, such that the instructions which execute on the computer or other programmable apparatus create means for implementing the functions specified in the flowchart block(s). These computer program instructions may also be stored in a computer-readable memory that may direct a computer or other programmable apparatus to function in a particular manner, such that the instructions stored in the computer-readable memory produce an article of manufacture which implements the functions specified in the flowchart block(s). The computer program instructions may also be loaded onto a computer or other programmable apparatus to cause a series of operations to be performed on the computer or other programmable apparatus to produce a computer-implemented process such that the instructions which execute on the computer or other programmable apparatus implement the functions specified in the flowchart block(s).

Accordingly, blocks of the flowchart support combinations of means for performing the specified functions and combinations of operations for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

In this regard, a method for planning the one or more ablation regions according to one example embodiment of the present invention is shown in FIG. 3, in which the one or more ablation regions are determined by the user manually. The method of FIG. 3 may entirely, except for operation 310, or at least in part, be executed automatically (e.g., without operator interaction to initiate each step or the series of steps) by processor 110.

The method comprises receiving vascular structure information of vessels inside a target region at operation 302. In an example embodiment, this operation is performed by the data receiver 201. The vascular structure information is for example in the form of angiography images, sometimes maybe by way of ultrasound, CT or MRI (for example, MFI image, and a kind of a contrast-based ultrasound image).

The method further comprises deriving a parametric map for the target region, based on the obtained vascular structure information at operation 304. In an example embodiment, this operation is performed by the parametric map deriver 202. A definition of such a parametric map has been described above in relation to FIG. 2 and will not be repeated here.

Additionally, the method may further comprise identifying one or more risky regions for ablative residual in the target region, based on the parametric map and a value threshold for a voxel to be in the risky region at operation 306. In an example embodiment, this operation is performed by the risky region identifier 204. One embodiment of identifying the risky regions has been described above in relation to the risky region identifier 204 in FIG. 2 and will not be repeated here.

Afterwards, at operation 308, the parametric map, additionally or alternatively the risky regions, will be shown to the user through UI 140, such as a viewer in a displayer. In an example embodiment, this operation is performed by the output controller 207.

Such a parametric map or risky regions, in combination with other information, such as tumor boundary, critical structures around the tumor, will help the user to determine one or more ablation regions at operation 310. Generally, the one or more ablation regions specified by the user need assessment to help the user to optimize his/her determination. The assessment takes place at operation 312 and this operation is performed by the assessor 205. One embodiment of assessing the one or more ablation regions has been described above in relation to the assessor 205 in FIG. 2 and will not be repeated here.

An indicator may be derived from the assessing result at operation 314 and fed back to the user via the UI 140 at operation 316. In an example embodiment, operation 314 is performed by the indicator deriver 206 and operation 316 is performed by the output controller 207.

A method for planning the one or more ablation regions according to another example embodiment of the present invention is shown in FIG. 4, in which the one or more ablation regions are determined by the processor 110 automatically. The method of FIG. 4 may entirely, except for operation 410, or at least in part, be executed automatically (e.g., without operator interaction to initiate each step or the series of steps) by processor 110.

The method comprises receiving vascular structure information of vessels inside a target region at operation 402. In an example embodiment, this operation is performed by the data receiver 201. The vascular structure information is for example in the form of angiography images, sometimes may be by way of ultrasound, CT or MRI (for example, MFI image, and a kind of a contrast-based ultrasound image).

The method further comprises deriving a parametric map for the target region based on the obtained vascular structure information at operation 404. In an example embodiment, this operation is performed by the parametric map deriver 202. A definition of such a parametric map has been described above in relation to FIG. 2 and will not be repeated here.

Additionally, the method may further comprise identifying one or more risky regions for ablative residual in the target region, based on the parametric map and a value threshold for a voxel in the risky region at operation 406. In an example embodiment, this operation is performed by the risky region identifier 204. One embodiment of identifying the risky regions has been described above in relation to the risky region identifier 204 in FIG. 2 and will not be repeated here.

Additionally, at operation 408, the parametric map, additionally or alternatively the risky regions, may be shown to the user through UI 140, such as a viewer in a displayer. In an example embodiment, this operation is performed by the output controller 207. However, such an operation displaying intermediate results is not necessarily required for the method in which the one or more ablation regions are determined by the processor 110 automatically.

Additionally, at operation 410, one or more user inputs regarding restrictions of the one or more ablation regions may be received. Such user inputs may define one or more of the following: the number of entry points, the position of the entry points, the number of total ablation regions, the number of ablations regions per entry point, etc. Thereafter, the one or more ablation regions will be determined, considering the received at least one user input.

Processor 110 determines one or more optimized ablation regions, based on such a parametric map or risky regions, in combination with other information, such as tumor boundary, critical structures around the tumor at operation 412. Generally, the optimization of the one or more ablation regions determined by the processor 110 requires assessment feedback, which in an example embodiment is provided by the assessor 205. One embodiment of assessing the one or more ablation regions has been described above in relation to the assessor 205 in FIG. 2 and will not be repeated here. In an example embodiment, operation 412 is performed by the position determiner 203 in combination with the assessor 205.

An indicator may be derived from the assessing result of the finally determined one or more ablation regions at operation 414 and displayed to the user via the UI 140 at operation 416. In an example embodiment, operation 414 is performed by the indicator deriver 206 and operation 416 is performed by the output controller 207.

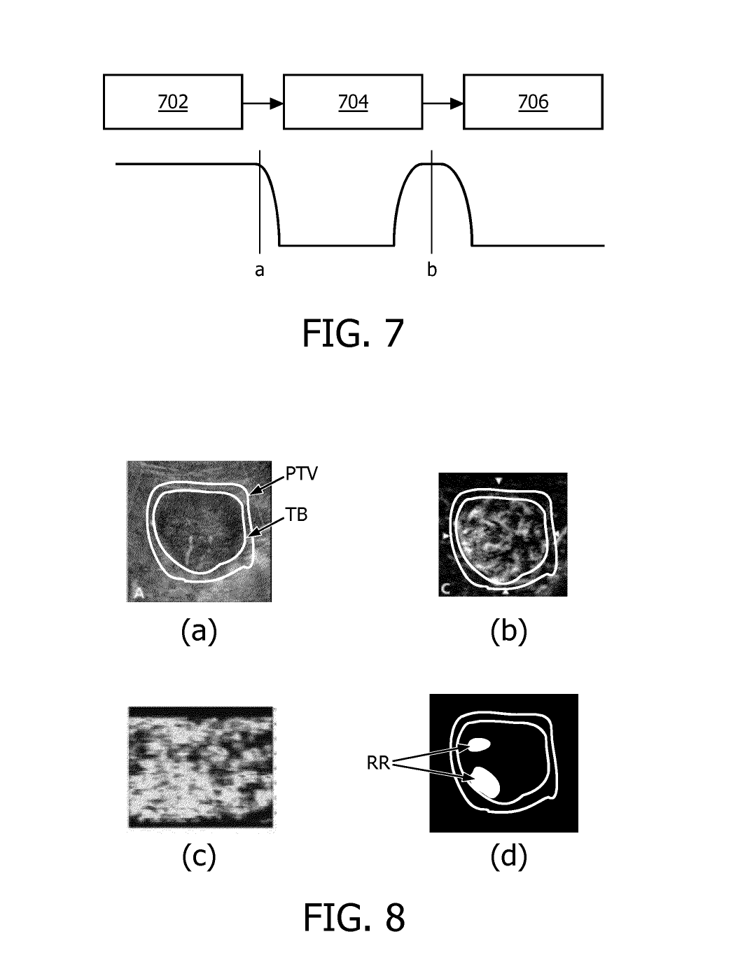

FIG. 7 illustrates a flowchart of imaging during ultrasound data acquisition, in accordance with one or more aspects set forth herein. Three types of imaging mode in an ultrasound system, i.e. conventional B-mode, conventional contrast-enhanced mode (CEUS) and contrast-enhanced replenishment mode, are sequentially performed to acquire sufficient and complementary information being used for assisting in the determination of one or more ablation regions for covering a target region to be ablated. An EM-tracked freehand sweep is similarly adapted over these three imaging modes for the purpose of 3-dimensional data reconstruction, implying that our thermal ablation planning according to one embodiment is a 3D solution and spatial transformation across all acquired images is essentially registered by the EM-tracking system. The imaging flow during ultrasound data acquisition mainly comprises the following steps:

(1) Sweeping in B-mode at block 702 with a normal high MI yields the anatomic structures around the ablation target. The risk of collateral damage, especially to the critical structures, is normally evaluated from the B-mode images, accounting for its large field of view.

(2) Sweeping during the artery phase at block 704. The primary tumor boundary is very sensitive in the artery phase of CEUS after the bolus injection (for example at point “a” of FIG. 7), which acts on the hyperechogenicity behavior in CEUS images. This information will leverage effective tumor identification, using a 3D segmentation tool, as the tumor boundary is quite clear in comparison with the one in the B-mode image.

(3) Sweeping in replenishment mode at block 706. The most important matter in one embodiment of this invention is the MFI data acquisition realized in replenishment mode. After an explosion at point “b” of FIG. 7, the intratumoral microvessels will be visible in an MFI image by means of single bubble tracking inside targeted vessels after a flash with high MI power. MFI plays a significant role in the embodiment of this invention because the advantage of tumor vascularity controlled residual as the output of planning fully relies on the quantitative vascularity features in MFI images.

The tumor boundary is outlined in the volumetric CEUS data by means of the applicable 3D segmentation tools, for instance Philips GeoBlend toolkit with the flexibility of user interaction. One example of 3D tumor boundary segmentation in CEUS is shown in FIG. 8 (a), where the segmented tumor boundary is denoted as TB. The tumor boundary with the uniformly dilated safety margin, normally 5 mm to 1 cm, which is considered necessary in order to kill microscopic cancer cells, is the planned target volume and is denoted as PTV in FIG. 8 (a). Herein, PTV is not only used to specify the tumor shape but also to define the ROI (region of interest) in the acquired MFI images for the vessel property quantification inside the tumor. The rationale behind PTV-defined ROI for intratumoral analysis is a well-configured EM-based registration during data acquisition. One example of tumor boundary mapped to MFI for ROI identification is shown in FIG. 8 (b).

The parametric map resulting from step 304 of FIG. 3 or step 404 of FIG. 4 is shown in FIG. 8 (c). The risky regions in relation to the tumor boundary with the uniformly dilated safety margin resulting from step 306 of FIG. 3 or step 406 of FIG. 4 is shown in FIG. 8 (d), indicated as “R.R.” in FIG. 8 (d), where, for example the IVP value is larger than 0.3.

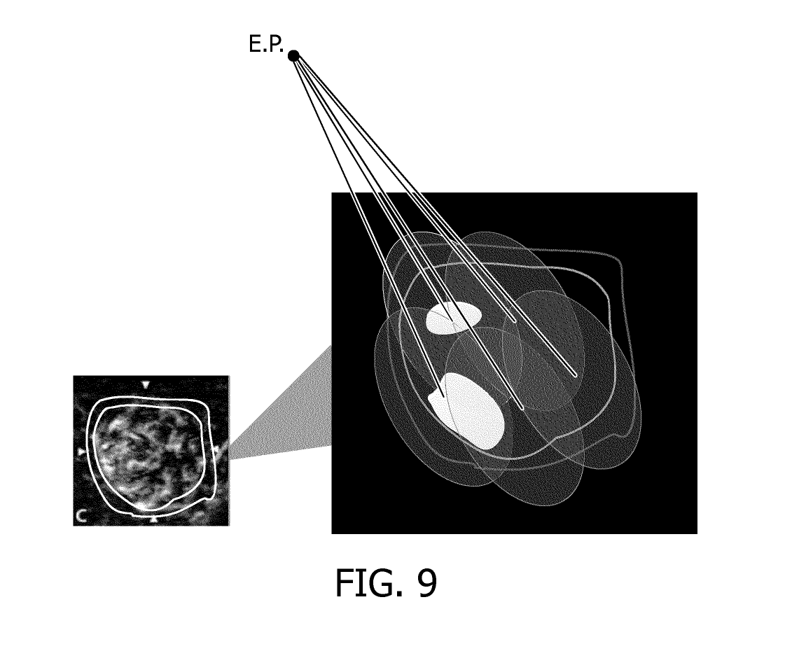

Finally, the composite ablations that fully cover the IVP risky regions are shown in FIG. 9, wherein “E.P.” is the entry point, and wherein correspondingly the residual is only left in the region that has the least angiogenesis severity. Those skilled in the art would appreciate that FIG. 9 illustrates one entry point and five elliptic ablation regions, yet the number of the entry points, and the number and shape of the ablation regions are not limited thereto.

While the embodiments have been illustrated and described herein, it will be understood by those skilled in the art that various changes and modifications may be made, and equivalents may be substituted for elements thereof, without departing from the true scope of the present technology. In addition, many modifications may be made to adapt to a particular situation and the teaching herein without departing from its central scope. Therefore, it is intended that the present embodiments not be limited to the particular embodiment disclosed as the best mode contemplated for carrying out the present technology, but that the present embodiments include all embodiments falling within the scope of the appended claims.

1. A device for assisting in a determination of one or more ablation regions for covering a target region to be ablated with a residual, comprising a processor configured to:
receive vascular structure information of vessels inside the target region; and
derive a parametric map for the target region, based on the received vascular structure information, each value in the parametric map indicating a metric of a corresponding voxel of the target region to be inside the one or more ablation regions.

2. The device according to claim 1, further comprising a first user interface configured to present the derived parametric map.

3. The device according to claim 1, wherein the processor is further configured to determine position of the one or more ablation regions, based on the derived parametric map.

4. The device according to claim 2, wherein the processor is further configured to:
identify one or more risky regions for ablative residual in the target region, based on the parametric map and a value threshold.

5. The device according to claim 4, wherein the value threshold is derived based on the parametric map and a predetermined ablation coverage rate.

6. The device according to claim 3, wherein the determination of the position of the one or more ablation regions is further based on one or more of:
part of the target region which is not covered by the one or more ablation regions;
part of the one or more ablation regions which is not covered by the target region; and
part of the one or more ablation regions which is overlapped with a predetermined critical region.

7. The device according to claim 2, further comprising a second user interface, wherein
the second user interface is configured to receive at least one of the following user inputs:
a user input for indicating a number or a maximum number of entry points for the one or more ablation regions;
a user input for indicating position of one or more entry points for the one or more ablation regions;
a user input for indicating a number or a maximum number of the one or more ablation regions;
a user input for indicating position of the one or more ablation regions; and
the processor is further configured to determine the position of the one or more ablation regions, based on the derived parametric map and the received at least one user input.

8. The device according to claim 1, wherein the processor is further configured to:
assess the one or more ablation regions, based on the derived parametric map;
derive an indicator from the assessing result; and
output the derived indicator via a third user interface.

9. The device according to claim 1, wherein the vascular structure information of the target region comprises an angiography image of the target region.

10. A method for assisting in a determination of one or more ablation regions for covering a target region to be ablated with a residual, comprising:
receiving vascular structure information of vessels inside the target region; and
deriving a parametric map for the target region, based on the received vascular structure information, each value in the parametric map indicating a metric of a corresponding voxel of the target region to be inside the one or more ablation regions.

11. The method according to claim 10, further comprising: determining position of the one or more ablation regions, based on the derived parametric map.

12. The method according to claim 10, further comprising:
identifying one or more risky regions for ablative residual in the target region, based on the parametric map and a value threshold.

13. The method according to claim 12, wherein the value threshold is derived based on the parametric map and a predetermined ablation coverage rate.

14. The method according to claim 1, wherein the determination of the position of the one or more ablation regions is further based on one or more of:
part of the target region which is not covered by the one or more ablation regions;
part of the one or more ablation regions which is not covered by the target region; and
part of the one or more ablation regions which is overlapped with a predetermined critical region.

15. A system for assisting in a determination of one or more ablation regions for covering a target region to be ablated with a residual, comprising:
an imaging component configured to generate vascular structure information of vessels inside the target region; and
a device in accordance with claim 1, the device being in communication with the imaging component.
